(19) United States
(12) Patent Application Publication
(10) Pub. No.: US 2021/0289855 A1
(43) Pub. Date: Sep. 23, 2021

(54) INFUSION FRIENDLY SWEATER DONNABLE WITH MEDICAL ATTACHMENTS

(71) Applicant: Aleah Wares, Inc., Midlothian, VA (US)

(72) Inventor: Aleah Colon-Alfonso, Sarasota, FL (US)

(21) Appl. No.: 17/316,105

(22) Filed: May 10, 2021

Related U.S. Application Data
(63) Continuation-in-part of application No. 16/544,370, filed on Aug. 19, 2019.

(51) Int. Cl. A41D 13/12

(52) U.S. Cl. CPC A41D 13/1281

(57) ABSTRACT

A clothing article accommodating pre-existing attire of a wearer and a pre-existing medical attachment, the article includes a first and second front side and a back side connected to a second front side, the article of clothing accommodating a pre-existing medical attachment connected to the wearer. The clothing article includes a bottom hem around the bottom and a collar around the top of the clothing article. A first front placket and a second front placket extend from the collar to the bottom hem. A first sleeve includes a lateral seam from the wrist to the first front placket, below the collar, with a top placket having fasteners and a bottom placket having mating fasteners. Donning or removing the clothing article includes opening the first lateral seam and wrapping the first sleeve around an arm of the wearer, closing the first lateral seam by engaging the fasteners with the mating fasteners.

### RELATED APPLICATIONS

The present application is a Continuation-in-part of and claims priority to U.S. patent application Ser. No. 16/544,370 filed Aug. 19, 2019.

### COPYRIGHT NOTICE

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### FIELD OF INVENTION

The disclosed technology relates generally to a patient's garment and more specifically to a supplemental garment for donning without disrupting medical attachments.

### BACKGROUND

Patients connected to medical devices can find themselves subjected to temperature fluctuations while undergoing lengthy routine procedures. For examples, procedures may be dialysis, chemotherapy, radiation, or the like. A patient is typically hooked-up to the medical device via an arm or thorax.

With these medical attachments, patients have limited options to help better regulate their own temperature. One approach is for the patient to dress for cooler temperatures and supplement with a blanket that can be laid over the patient. Because the patient is physically connected with medical attachments, the patient cannot pull a normal sweater over themselves. The same problem occurs with sleeves and medical attachments getting caught in the sleeves.

One existing solution is the MIGHTY WRAP™ available from Mighty Well, Inc. of Newport, R.I. This product is a very large and roomy open-front sweater with large arm sockets. This solution suffers from the same complications as other prior art solutions, the wearer must either snake medical attachment connections through the front opening of the wrap or don the product prior to medical attachment and keep the wrap on during the whole procedure.

Additionally, this solution risks dislocation or disruption of the IV or other attachment.

There are several known hospital gown solutions. These all suffer from the same deficiency of being directed to a primary gown to cover a patient, not providing supplementation for temperature regulation. For a hospital gown, the patient removes all underlying clothes and the gown is about access to the patient's body, not accounting for comfort and temperature regulation.

U.S. Pat. No. 4,570,268 (“the '268 patent”) is a three-piece patient garment with various fasteners extending down the arms and across the front. The '268 patent hospital garment is the sole article of clothing worn by the patient, and it includes short sleeves, its three-piece construction allows for medical-access to the patient. The access is for in-hospital medical procedures, such as allowing for x-rays, thorax and abdomen examinations, even a nursing mother access to breastfeed. Nothing in the '268 patent provides for temperature regulation or supporting/comforting a wearer while undergoing medical procedures. Moreover, the '268 patent gown is donned by the patient prior to undertaking any medical procedures, with the various fasteners defining later medical doctor access.

U.S. Publication No. 2013/0276202 (“the '202 Pub.”) teaches a front-opening hospital gown with overlapping front panels. The '202 Pub. hospital gown teaches fasteners along a portion of the sleeves, where opening the fasteners allows medical access to the arms. This '202 Pub. gown is a typical high-use medical gown designed as the primary patient covering garment and able to be subjected to a large number of high intensity washings. This gown does not seek providing comfort to the patient. The '202 Pub. gown is very similar to the '268 patent gown in allowing ease of doctor access to the patient wearing only the gown itself.

U.S. Publication No. 2015/0157069 (“the '069 Pub.”) teaches a patient gown similar to the '202 publication and the '268 patent. This gown is a three panel hospital gown, donned on the patient without any additional clothing, with the fastener locations granting access to the patient. Because the '069 Pub. gown is a primary clothing article, the fasteners are made with higher stability fasteners to survive the repeated high temperature washings and the gown is designed to include privacy for the otherwise-naked patient.

These existing solutions all fail to provide donning and removal options for the patient while attached to one or more medical devices, in conjunction with allowing the patient to wear his or her own clothing. The wrap solutions bunch or otherwise constrict existing medical connections. The hospital gown solutions are not meant for comfort to the patient based on being a hospital gown, are not directed to donning and removal options during treatment because the gown is worn prior to commencement of any procedure, and these existing solutions including design complications inhibiting a wearer to don and remove the clothing without additional assistance.

As such, there exists a need for garment, an infusion friendly sweater, that accommodates medical attachments to the wearer and allows for quick and easy one-handed donning and removal of the infusion friendly sweater due to temperature fluctuations.

### BRIEF DESCRIPTION

An infusion friendly sweater serves as a secondary article of clothing as it supplements existing attire of a wearer already connected to and having pre-existing medical attachments. The infusion friendly sweater is an article of clothing that includes a first front side, a second front side, and a back side. The article of clothing includes a bottom hem extending around bottom portions of the first and second front sides and the back side. The article of clothing includes a collar extending from top portions of the first and second front side, and the back side.

The first front side includes a first front placket. The second front side includes a second front placket. The plackets extend from the collar to the bottom hem.

The article of clothing includes a first sleeve with a first lateral seam allowing for donning and removal without disruption of the pre-existing medical attachments. The lateral seam extends from a wrist hem, up the arm, and then across the first front portion, to the first front placket.

The first lateral seam includes a top placket with fasteners and a bottom placket with mating fasteners.

In the event the wearer has her arm connected to a pre-existing medical attachment, e.g. an IV line, the combination of plackets allow for donning the clothing article without removing or disconnecting her given intravenous treatment. The wearer can slide the non-connected arm through a second sleeve and wrap the back side of the clothing article around her torso.

With the lateral seam open, the first sleeve can be wrapped around the wearer arm while remaining attached to the medical line. Engagement of the fasteners and mating fasteners secures the sleeve article in place.

The positioning of the fasteners and mating fasteners generate a medical attachment channel, where the medical attachment safely passes through the closed first lateral seam, between the top and bottom plackets.

The article of clothing further includes a lateral seam closure disposed either on the first front placket or the first front side. A corresponding lateral seam connector is disposed on the opposing side of the lateral seam. For instance, if the closure is below or on the bottom placket of the first lateral seam, the connector can be disposed on the top placket, the collar, or other position above the lateral seam. The lateral seam closure engages the connector for an additional secure donning of the clothing article. The lateral seam closure additionally facilities donning the clothing article without disrupting the medical attachment.

The first front portion includes a first front side placket, which can include a plurality of front fasteners therein. The second front portion includes a second front side placket, which can include a plurality of front mating fasteners. Similar to the lateral seam noted above, the wearer can secure the clothing article around her front portion by engaging the front fasteners with front mating fasteners.

In one embodiment, the first sleeve may include a wrist pull tab at the wrist hem. The wrist pull tab extends outward from either the top placket or the bottom placket, having a tab fastener thereon. A wrist tab mating fastener is disposed on the wrist hem on the opposing placket. Whereby the wearer includes an additional means for donning the clothing article by securing the tab fastener to of the wrist pull tab to the wrist tab mating fastener.

Where one embodiment is the first sleeve having the lateral seam, the second sleeve of the clothing article can also include a second lateral seam. In another embodiment, the second sleeve can be a closed, normal, sleeve. In this case, the wearer slides the free arm, e.g. the arm not connected to a medical attachment, through the sleeve and wraps the open sleeve around the medical attachment.

Further embodiments may include one or more internal pockets within the sweater. The pockets can include lining or other material for accommodating a temperature adjustment packet. For example, the pocket may include lining with sufficient structure to support holding an ice pack and water-resistant material to accommodate any melting or condensation from the pack. Another can be inclusion of a lining to accommodate a heat pack with material to defuse the warm temperature or guard against heat sensitivities. Another embodiment could employ a clear pocket to allow for simple and discreet external monitoring of external medical devices connected to the wearer, such as surgical drains.

In another embodiment, the clothing article can include wiring for connecting at least one electrical connection. A connector may include a biofeedback or measurement sensor, such as a heart rate monitor, and/or a body temperature sensor, by way of example. A connector can include electronic devices, such as speakers, headphone connections, pulse oximeters, or other devices.

The clothing article can additionally include interior channel(s) allowing for airflow. The disposition of channels could allow for connecting external airflow device(s) to accommodate blowing air of different temperatures.

In one embodiment, the article of clothing can additionally be reversible. By being reversible, the clothing article has a closed, e.g. normal, sleeve, and the open seam sleeve. For instance, a default open sleeve having the lateral seam, can be right arm for wearers who have medical attachments primary to their right arm. If the wearer has a left arm connection, reversing the clothing article thus allows for donning and removing the sweater to now accommodate a left-arm connection.

Additionally, the fasteners can be any device or devices for donning the sweater, such as but not limited to magnets, buttons, snaps, hook-and-loop connectors, etc., or a combination thereof.

The medical attachments can be any suitable attachment to the person. Examples can include intravenous (IV) lines, peripherally inserted central catheters, subcutaneous ports, central venous catheters, a dialysis connection, etc.

Therein, the infusion friendly sweater, via the multiple plackets and the pull tab(s), allows for donning over pre-existing attire by a wearer having pre-existing medical attachments. The infusion friendly sweater is worn over the wearer's pre-existing clothing and can be made of soft, comfortable material to help regulate body temperature. The infusion friendly comfort sweater allows a wearer undergoing a medical procedure, such as an infusion, to self-regulate and better adjust to temperature fluctuations, by donning and removing the sweater without adversely impacting the connected medical attachments.

### BRIEF DESCRIPTION OF THE DRAWINGS

FIG. 1 illustrates a front view of an infusion friendly sweater with the fasteners engaged;

FIG. 2 illustrates a front view of the infusion friendly sweater with the fasteners disengaged, the sweater fully open prior to donning;

FIG. 3 illustrates a partial view of the sweater being donned around a wearer's arm;

FIG. 4 illustrates a partial view of the sweater being donned around a wearer having a medical attachment;

FIG. 5 illustrates a partial view of the donned sweater with the wearer having a medical attachment;

FIG. 6 illustrates a partial front view of the collar tab of the sweater;

FIG. 7 illustrates a partial back view of the collar portion of the sweater;

FIG. 8 illustrates a partial front view of the collar tab engagement on the sweater;

FIGS. 9a-9b illustrate partial back views of the collar tab engagement on the sweater;

FIG. 10 illustrates a partial top view of the sweater donned without disruption of a medical attachment;

FIG. 11 illustrates an article of clothing having a lateral seam extending to a front placket; and

FIG. 12 illustrates the article of clothing of FIG. 11 with the lateral seam in the open position.

A better understanding of the disclosed technology will be obtained from the following detailed description of the preferred embodiments taken in conjunction with the drawings and the attached claims.

### DETAILED DESCRIPTION

As described herein, a wearer undergoing medical treatment can better regulate body temperature using the infusion friendly sweater. The infusion friendly sweater, through its unique construction allows for donning and removal without disruption of existing medical attachments, e.g. catheter, IV line(s), etc. The infusion friendly sweater further allows donning and removal individually by the wearer using a single-hand where the other hand is rendered immobile from the medical attachment(s).

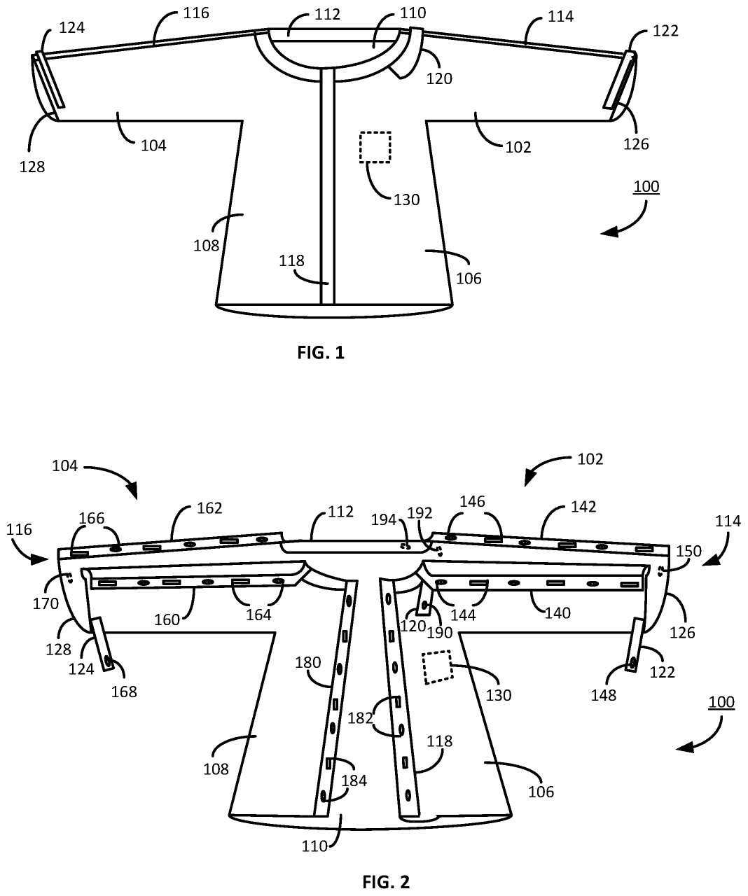

FIG. 1 illustrates a front view of one embodiment of an infusion friendly sweater 100 in a closed position. The sweater 100 includes a first arm portion 102, a second arm portion 104, a first front portion 106, a second front portion 108, a back portion 110, and a collar portion 112. The arm portions 102, 104 include plackets 114, 116. The first front portion 106 include a placket 118. The second front portion 108 also includes a placket, not visible as being occluded in FIG. 1 by the placket 118.

A placket is an opening on a piece of clothing allowing for donning and removal of the clothing item. The placket can include attachments for opening/closing the placket, for example buttons and button-holes or snaps found on plackets on a blouse or dress shirt. Plackets can be functional and/or decorative.

The infusion friendly sweater 100, by having multiple plackets at the arms and down the front, allows for donning and removal without disruption of medical attachments connected to a wearer.

The infusion friendly sweater 100 includes a collar pull tab 120, a first wrist tab 122 and a second wrist tab 124. The first wrist tab 122 is disposed at a first wrist portion 126 of the first arm portion 102, the second wrist tab 124 is disposed at a second wrist portion 128 of the second arm portion 104.

While not visible in FIG. 1, the plackets 114, 116, 118 include fasteners and mating fasteners. As used herein, fasteners and mating fasteners provide for interconnection to secure the placket closed. A fastener and mating fasteners are complimentary elements. Fasteners and mating fasteners can include magnets, buttons and button-holes, hook-and-loop fasteners, zippers, clasps, or any other suitable fastener, or a combination of different fasteners, as recognized by one skilled in the art.

The infusion friendly sweater 100 includes the collar pull tab 120 that additionally allows securing the sweater 100 around a wearer. The wrist pull tab 122 and/or 124 additionally secures the sweater 100 in place.

One embodiment may include one or more interior or hidden pockets, such as pocket 130. Additionally, the sweater 100 may include exterior pockets (not shown), including pockets for placing hands therein, or pocket(s) for carrying items. For example, the sweater 100 may allow an interior pocket 130 for carrying a medical device or accoutrements associated with the medical attachment. The pocket or pockets can also include heat packets or cool packets to further effectuate temperature adjustment of the wearer.

Where FIG. 1 illustrates the closed sweater 100, FIG. 2 illustrates the sweater 100 in an open position. In FIG. 2, the sweater 100 is in a position for being donned around a wearer having connected medical attachments.

FIG. 2 illustrates the sweater 100 with the first arm portion 102, second arm portion 104, first front portion 106, second front portion 108, back portion 110, and collar portion 112. When open, the first arm portion 102 includes a top placket 140 and a bottom placket 142. The top placket 140 includes a plurality of fasteners 144 with the bottom placket 142 including mating fasteners 146. Additionally, the wrist tab 122 includes a fastener 148 with a mating fastener 150 embedded in either arm portion 104 or the wrist portion 126.

In one embodiment, the second arm portion 104 includes similar construction with a top placket 160 and bottom placket 162. The top placket 160 includes fasteners 164 and the bottom placket 162 has mating fasteners 166. The wrist tab 124 includes a fastener 168. A corresponding mating fastener 170 is in either the arm portion 102 or the wrist portion 170.

In the front, the first front portion 106 has front placket 118 and the second front portion 108 has an interior placket 180. The front placket 118 has a plurality of spaced-apart fasteners 182 and the interior placket 180 has mating fasteners 184.

The collar pull tab 120 is shown in an open position, with fastener 190. A mating fastener 192 and/or 194 can be disposed on the back portion 110 or the collar portion 112 such that when attached it further secures the sweater 100 around the wearer. The collar pull tab 120 also has an extended length allowing for connecting the fastener 190 and mating fastener 192 or 194 using one hand in the event the other hand is immobile.

As noted above, the fasteners and mating fasteners on the plackets are spaced apart. The plackets allow for wrapping the sweater around the wearer and securing the sweater in place by connecting the fasteners and mating fasteners. The pull tab(s) secure(s) the sweater in place when the wearer has limited mobility. Additionally, the donning and removal of the sweater is over the wearer's existing clothing. The sweater is a supplemental garment allowing the wearer to keep on existing clothing and add/remove the sweater solely for comfort purposes.

The spaced apart nature of the fasteners accommodates pre-existing medical attachments. By having spacing between fasteners, the placket naturally creates an opening accommodating the attachments.

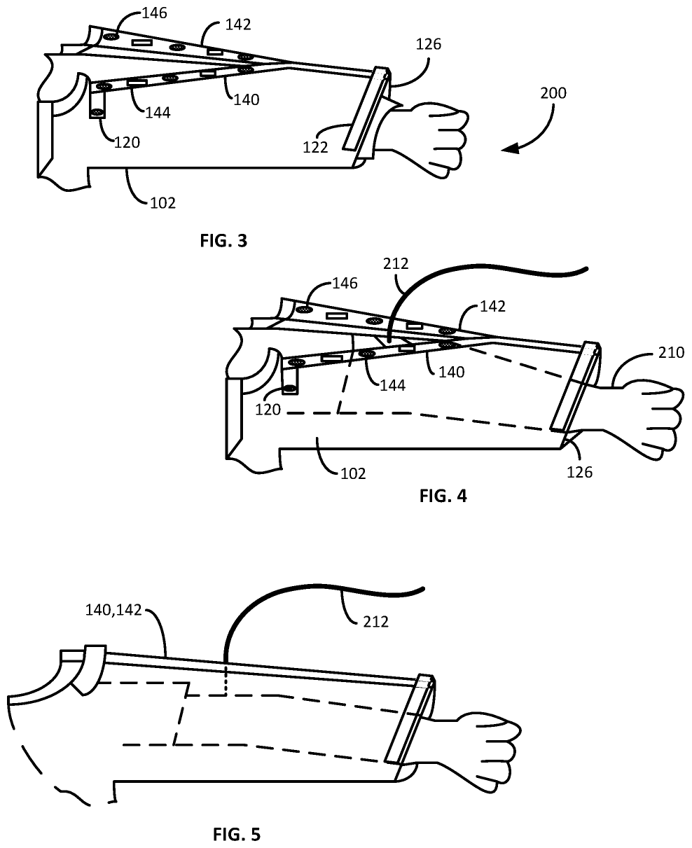

FIG. 3 illustrates a partial view of a wearer 200 donning the first arm portion 102 of the sweater 100. Here, the wearer 200 has on regular clothes such as a long-sleeved shirt. In FIG. 3, the arm is not connected to any medical attachments, but donning the sweater can include sliding the arm through the wrist portion 126 opening or wrapping the arm portion 102 around the wearer's arm and closing the fasteners 144 and mating fasteners 146 on the plackets 140, 142. Also visible, the wrist tab 122 is secured in place and the collar tab 120 is open.

FIG. 4 illustrates a wearer arm 210 with a medical attachment 212 connected thereto. As noted above, the medical attachment 212 can be any suitable device or devices connected to the wearer, such as a catheter, IV line, electrical monitoring connection(s), etc.

With prior solutions, as the wearer inserted his or her arm into the garment, the attachment 212 would be caught within the interior of the sleeve. Or, in the alternative, the wearer would have to remove the attachment 212, the arm slid through the arm socket, and then the attachment 212 re-attached.

By contrast, the wearer secures the arm portion 102 of the infusion friendly sweater around the wearer's arm 210 by selectively connecting fasteners 146 and mating fasteners 148 along the plackets 140, 142. For clarity purposes, FIG. 4 illustrates the fasteners and mating fasteners already engaged on the half of the arm portion 102 nearer the wrist portion 126.

As the plackets 140 and 142 are connected, FIG. 5 illustrates a front view of the infusion friendly sweater fully donned around the wearer's arm 210. As illustrated, the medical attachment 212 remains undisturbed, extending through an offset opening made possible by the spaced-apart fasteners and mating fasteners from the plackets 142, 144.

The infusion friendly sweater also includes a collar tab assisting with single-handed donning and removal. The wearer is typically connected to one or more machines. These medical treatments can take an extended period of time, where the wearer has limited mobility and can find themselves subject to temperature fluctuations. For example, during a chemotherapy treatment, the wearer sits for an extended time period with limited mobility because of an IV line. Some infusions can last upwards of 12 or more hours, requiring the wearer to be medically tethered to the IV line in a generally immobile environment.

These medical treatment sessions are often performed with minimal medical supervision, such as a nurse or assistant periodically checking in on the wearer. Thus, the infusion friendly sweater allows for donning the sweater without outside assistance. With the wearer having limited mobility, the collar pull tab allows the wearer to secure the sweater in place using a single free hand. This is especially important given the often sudden temperature fluctuations patients experience during intravenous treatments.

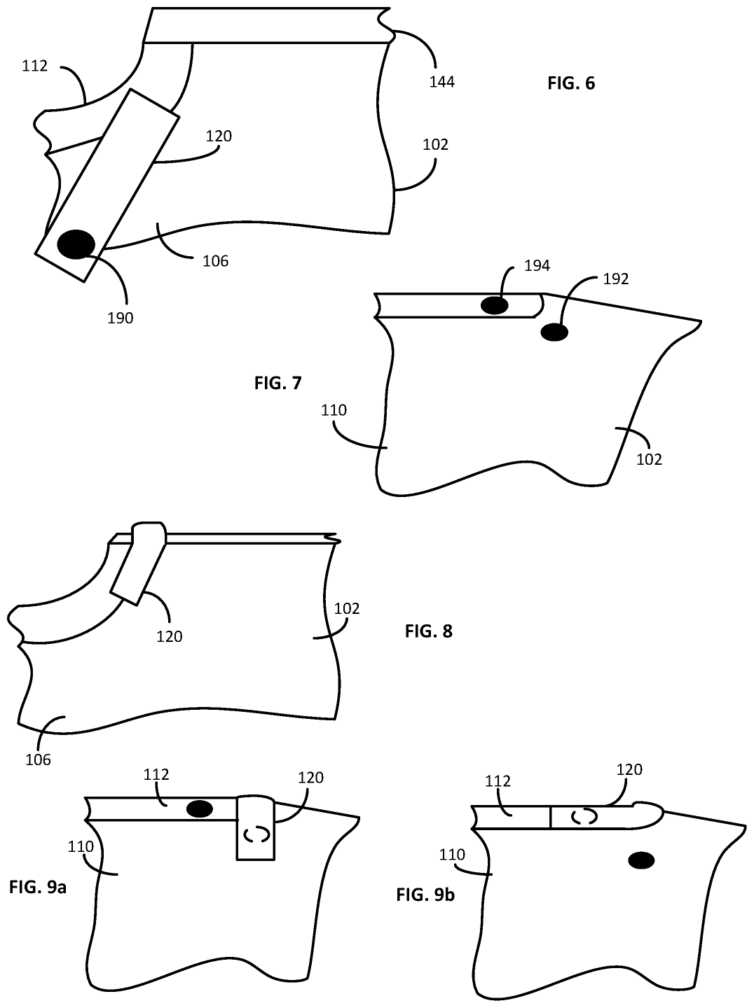

FIGS. 6-9b illustrate one embodiment of the collar pull tab. FIG. 6 is a partial view of the arm portion 102 with the collar pull tab 120 in an open position, e.g. not secured around the wearer. The fastener 190 is disposed on a distal end of the tab 120. The pull tab 120 is disposed, in this exemplary embodiment, connected to the front portion 106. It is recognized the pull tab 120 may be affixed to the arm top placket 144, the arm portion 102, or even the collar portion 112. The specific location of the tab 120 connected to the sweater can be dependent on the length of the tab itself.

Where FIG. 6 is a front view, FIG. 7 is a rear view of the infusion friendly sweater with the arm portion 102 and the back portion 110. Visible in FIG. 7 are two exemplary mating fasteners 192, 194. The mating fasteners 192, 194 are for receipt and mating with the fastener 190 of FIG. 6 when the tab is secured in place.

FIG. 7 illustrates the alternative embodiments of various mating fastener locations 192, 194. The mating fastener 220 can allow for more of an open front position of the sweater when secured and the mating fastener 222 can create a modified turtleneck structure. A mating fastener may also be disposed on the arm portion, for example if the wearer has limited mobility and unable to reach over up to the collar or back portion.

FIGS. 8-9b illustrate front and rear views in a closed position. FIG. 8 illustrates the front portion 106 with the collar pull tab 120 pulled up and around the arm portion 102. FIGS. 9a-b illustrate the rear view showing the pull tab secured in place. FIG. 9a uses the mating fastener on the back portion 110. FIG. 9b uses the mating fastener on the collar portion 112.

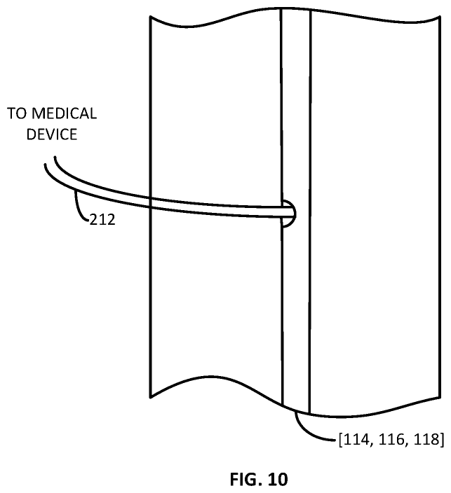

For further clarity, FIG. 10 illustrates a top view of a closed placket. The placket may be an arm placket 114 and 116. The placket can also be a front placket 118. FIG. 10 illustrates the connecting of the fasteners and mating fasteners does not adversely affect the medical attachment 212. Therefore, the wearer being already connected to medical device(s) through the attachment(s) 212 can readily don and remove the infusion friendly sweater.

The infusion friendly sweater may include varying embodiments offering varying degrees of options for the wearer. For example, FIGS. 1-2 illustrate one embodiment having multiple plackets allowing for fully wrapping the sweater around the wearer prior to closing the plackets.

In another embodiment, the sweater may have a single fastener/mating fastener placket on one arm, with the other arm being a standard closed channel found in a standard sweater. In this embodiment, the wearer has medical attachments to one arm, the other arm is free to slide through the wrist opening, leaving the arm with the open plackets to be wrapped around the wearer and the medical attachment.

As noted above, the front portion may close using plackets with fasteners and mating fasteners. But further embodiments may use other types of closures, such as extra material to fold over, string or ties to tie up the front for examples.

The infusion friendly sweater, not being a primary medical clothing item, e.g. a hospital gown, is not subject to manufacturing specifications directed to repeated high-temperature washing. The infusion friendly sweater can be made of any suitable material allowing for comfort to the patient, such as soft cotton, wool, synthetic materials, blends of different materials. The infusion friendly sweater can be made of a material designed to retain heat in addition to the comfort. For example different materials can promote heat retention, for example wool, thicker cotton, synthetic materials, etc. The sweater may also include inserts or inner layers of material to further promote temperature regulation.

In varying embodiments, the sweater may include designs on the material or attached to the material. For example, for a child sweater, the infusion friendly sweater may include pictures of animals. In another example, for a child the sweater may include hook-and-loop fasteners for attaching a stuffed animal or plush toy to the outside or an exterior pocket for holding the animal.

One embodiment may include pockets for holding additional equipment associated with the wearer's medical treatment. These pockets can also hold personal items. Pockets can be internal and/or external pockets, positioned to assist the wearer without interfering with its ease of donning and removal.

Further embodiments may include internal pockets or channels for circulating airflow. For example, various pockets can be located at different areas/regions of the sweater. In one example, pockets near the front portions can hold hot or cold packets to help temperature regulation at the wearer's torso. In another embodiment, pocket(s) can be within the arm portion for assisting with discomfort of the medical attachments, such as a pocket to hold a cold packet near the insertion point of the medical attachment.

The sweater may include pockets or channels connectable to an external air flow generator that sends cooled or heated air for internal circulation within the sweater. For example, an air circulation device can generate cooled or heated air, and upon insertion into an interior channel within the sweater, the cool or heated air can circulate to better comfort the wearer. The interior channel may include a channel formed by the closing of the plackets.

Moreover, as the infusion friendly sweater is a secondary or supplementary clothing article, the sweater can use larger dimensions for draping over the wearer. For example, the sweater can be designed with larger proportions from a standard sized sweater, allowing for roomier fitting and more draping over the wearer.

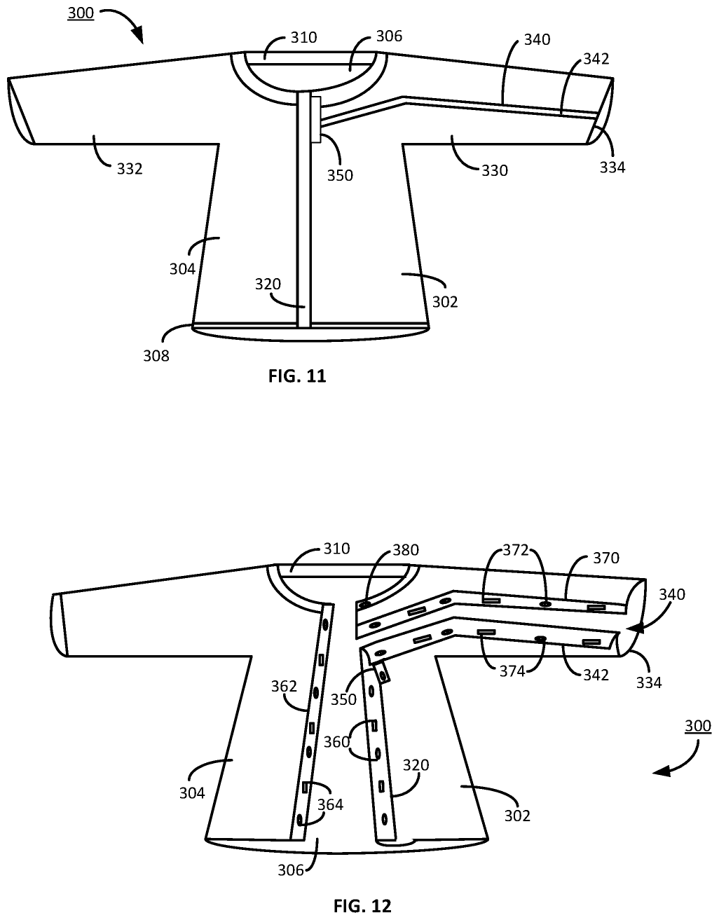

In a further embodiment, the infusion friendly sweater is an article of clothing as illustrated in FIGS. 11 and 12. The article of clothing 300 includes a first front side 302, a second front side 304, and a back side 306 (not fully visible).

The clothing article 300 includes a bottom hem 308 disposed at a bottom portion of the first and second front sides 302, 304, and a bottom portion of the back side 306. The bottom hem 308 extends around the bottom edge of the clothing article 300.

The clothing article 300 includes a collar 310. The collar 310 is disposed at a top portion of the first and second front sides 302, 304, and a top portion of the back side 306. The collar 310 extends around the top edge of the clothing article 300.

The clothing article 300 includes a first front placket 320 and a second front placket (not visible in FIG. 11). The first front placket 320, when the clothing article 300 is in a closed position, covers the second front placket. The first front placket 320 and the second front placket extend from the bottom hem 308 to the collar 310.

The clothing article 300 includes a first sleeve 330 and a second sleeve 332. Where FIG. 11 illustrates the first sleeve as the left sleeve, this is merely an illustration and not a requirement. The first sleeve 330 includes a wrist hem 334 at the distal end. For clarity, when a user wears the clothing article 300, the wrist hem 334 may be positioned in correlation with the user's wrist.

In this embodiment, the first sleeve 330 includes a lateral seam 340. The lateral seam 340 runs from the wrist hem 334 to the first front placket 320, extending below the collar 310.

The first lateral seam 340 includes a bottom placket 342 and a top placket. In the closed seam 340, the bottom placket 342 occludes the top placket from view. As described in greater detail below, the bottom placket 342 and top placket can be separated or mated.

Further visible in FIG. 11, a lateral seam closure 350 is disposed on one side of the lateral seam 340. Not visible, a lateral seam connector, is disposed on the opposing side of the lateral seam 340. The lateral seam closure 350 is a piece of fabric or other material that extends out from clothing article, such as a strap. The closure 350 may include a connector element. The lateral seam connector has a mating element that matches up with the closure 350. For example, the closure 350 may be a strap with hook-and-loop connectors and the connector element is a hook pad that secures the closure 350 in place.

FIG. 11 is the clothing article in a closed position. This may be akin to how it is worn by a user connected to a pre-existing medical condition. By contrast, FIG. 12 illustrates the article 300 in its open position for donning (or removal) by the wearer. The first front side 302 includes the first front placket 320. Visible in FIG. 12 are a plurality of front fasteners 360. The second front side 304 includes the second front placket 362 with a plurality of front mating fasteners 364.

Opening of the front placket 320, 362 allows for the user to wrap the first front side 302, second front side 304, and back side 306 around her torso. Engagement of the front fasteners 360 with front mating fasteners 364 secure the first front placket 320 to the second front placket 362. The article 300 covers the torso of the wearer, so the bottom hem 308 should sit near or around the waist of the wearer.

To accommodate pre-existing medical attachments connected to an arm of the user, FIG. 12 illustrates the lateral seam 340, on the first sleeve 330, in an open position. Visible in lateral seam 340 is the top placket 370 having a plurality of fasteners 372, as well as the bottom placket 342 having a plurality of mating fasteners 374. The lateral seam 340 extends from the wrist hem 334 to first front placket 320, extending across the first front side 302, below the collar 310.

Prior garment solutions have openings running across the shoulder to the collar, those solutions allow for limited medical access. Those prior solutions are about a disposal hospital garment for patient modesty and healthcare access. Whereas the inclusion of the lateral seam 340 extending from the wrist hem 334 to the first front placket 320 allows for the wearer to readily don or remove the clothing article 300 relative to existing clothing, and this donning or removal fully accommodates existing medical attachments. The spacing and engagement of the mating fasteners 374 to the fasteners 372 in the lateral seam 340 thereby creates a medical attachment channel allowing for pre-existing medical connections, such as noted in FIG. 10 above.

Further visible in FIG. 12, the lateral seam closure 350 is in the open or disengaged position. A corresponding lateral seam connector 380 is above the lateral seam 340. FIG. 12 illustrates the connector 380 at the collar 310, but this can be positioned at any location on the article 300 on the opposite side of the seam 340 from the closure 350. The closure 350 engages the connector 380 to further secure the lateral seam 340 in its closed position. Whereas, the closure 350 can also be a pull tab to assist in opening the lateral seam 340 by separating the mating fasteners 372 and fasteners 374.

Where the present clothing article 300 allows for donning and removal, the lateral seam 340 further allows for the user to don or remove the article 300 by herself. When attached to a medical attachment, such as an IV line into the arm, the patient may wish to don the sweater without requiring assistance from a nurse or other caretaker. The lateral seam 340 allows the user to wrap the first sleeve 330 around her arm and manually close the seam to accommodate the medical attachment. The displacement of the seam 340 to the front placket 320 better accommodates the wearer, including using the lateral seam closure as a handle to hold the clothing article 300 and begin alignment for closing the seam 340 on the sleeve 330.

Whereby, the positioning of the lateral seam 340 from the wrist hem 334 to the front placket 320, below the collar 310, allows for a user to individually don or remove the sweater. The positioning of fasteners and mating fasteners on the top and bottom plackets of the lateral seam accommodates pre-existing medical attachments to an arm of the wearer, facilitating donning or removal without disrupting or risking dislodgement of the attachment. In this embodiment, the position of the seam 340 extending to the front placket 320 below the collar 310 places the seam aligned with an interior portion of the arm of the wearer, the seam more aligning with a crux of the elbow of the wearer and commonly accessed venous entry points such as the cephalic and basilic veins. This seam alignments on the interior of the sleeve 330 better accommodates intravenous medical attachments, improving user comfort while wearing the article 300, but also better ensuring against accidental dislodgement of the connector.

The clothing article 300 may include a single lateral seam or lateral seams for each sleeve. For instance, one embodiment can include a first lateral seam on the first sleeve and a second lateral sleeve on the second sleeve. In this embodiment, a user connected to a medical attachment in one arm can don the connected arm by wrapping the open seam around the arm, donning the opposing arm by having the seam already closed and sliding her arm down the arm hole.

In another embodiment, the clothing article 300 may be fully reversible. In this embodiment, having a lateral seam on only one sleeve still allows the user to be connected to a medical attachment in either arm. For example, if the lateral seam is on the left arm and the user has an IV attachment on her right arm, the clothing article 300 may be turned inside out, thus making the lateral seam now on the right arm. Therefore, in this embodiment plackets and fasteners/mating fasteners can be inverted or accessible on both sides of the placket. For example, if the fasteners and mating fasteners are magnets, the magnetic connection occurs both in an original clothing format and a reversed format. In another example, the fasteners and mating fasteners may be disposed on both sides of each placket.

The clothing article 300 can resemble other known clothing articles, with the inclusion of the lateral seam and other modifications noted herein. For example, the FIGS. 11 and 12 article resembles a pull-over or a jacket, having the collar 310 mate around the neckline. Variations can include a hooded version having a hood connected to the collar 310 at the back side 306. Another variation can resemble a cardigan where the collar 310 does not necessarily connect at the neckline, but blends more evenly into the front plackets.

The present clothing article may include additional embodiments facilitating improved user benefits. For example, one embodiment may include one or more internal pockets. The pocket or pockets can be disposed at various locations assisting the wearer. One pocket embodiment may include lining or other fabric accommodating heating or cooling packets. For example, a temperature dispersing material can be lined within the pocket to ensure a heat packet warms, or a cold packet cools, the wearer, but does not have a direct impact on the wearers skin. Similarly, the pocket(s) may include lining for a cool packet to avoid condensation or liquid dispersion as the cool packet warms up.

Further embodiments may include wiring or accommodating sensor(s) for the wearer. Wiring can include internal wiring embedded with the article to accommodate the existing medical attachment. One example can be headphones. The user is connected to an IV line, any additional external wire(s) or cable(s) can cause problems, such as getting snagged or wrapped on the IV line. Internal wiring can connect headphones for the wearer, securing the wiring within or to the clothing to ensure against entanglement with the medical attachment. This wiring could be strategically placed, for example headphones sewn into the collar 310 to provide the sound of music close to the wearer's ears.

The clothing article improves connectivity and accommodates medical or feedback devices. For example, the user may employ one or more wearable devices, such as a biofeedback sensor device. Examples may include, but are not limited to, a temperature measurement device, an oxygen saturation measurement device, a heart rate sensor, a portable EKG/ECG monitor, among others. The clothing article can include pockets or connectors for the sensor devices. As noted above, a big concern is tangling anything with the IV line and possibly ripping out the IV connection. Thus, the clothing article further accommodates the wearer by protecting the IV line connection.

Another embodiment may include lightweight wireless speakers affixed or sown into the clothing article. One embodiment can include small, lightweight speakers within the collar.

In another embodiment, the clothing article 300 may include further attachment(s) elements at the wrist. For example, wrist tab 122, and related elements, of FIG. 2 can further assist with open or closing the lateral seam 340.

In one embodiment, the clothing article can be composed of an anti-microbial material. The clothing article itself can be all one material or in another embodiment the areas that come into contact with injection sites, port lines, or other sensitive locations, can be made of this anti-microbial material. For example, the back and front sides of the article can be made of a different material from the sleeve(s), or in another embodiment the plackets are made of an anti-microbial material with the rest of the article in a more plush or softer fabric.

Herein, the clothing article 300 improves of pre-existing hospital garments with open sleeves for medical access. The inclusion of the lateral seam extending to the front placket improves donning/removing by the user, as well as the position of the seam ensures against discomfort or dislodgement of the medical attachment. The wearer can, without additional assistance, don or remove the clothing article whilst connected to a medical device, safeguarding the medical attachment but also quickly and personally attending to her comfort.

FIGS. 1 through 12 are conceptual illustrations allowing for an explanation of the present invention. Notably, the figures and examples above are not meant to limit the scope of the present invention to a single embodiment, as other embodiments are possible by way of interchange of some or all of the described or illustrated elements. Moreover, where certain elements of the present invention can be partially or fully implemented using known components, only those portions of such known components that are necessary for an understanding of the present invention are described, and detailed descriptions of other portions of such known components are omitted so as not to obscure the invention. In the present specification, an embodiment showing a singular component should not necessarily be limited to other embodiments including a plurality of the same component, and vice-versa, unless explicitly stated otherwise herein. Moreover, Applicant does not intend for any term in the specification or claims to be ascribed an uncommon or special meaning unless explicitly set forth as such. Further, the present invention encompasses present and future known equivalents to the known components referred to herein by way of illustration.

The foregoing description of the specific embodiments so fully reveals the general nature of the invention that others can, by applying knowledge within the skill of the relevant art(s) (including the contents of the documents cited and incorporated by reference herein), readily modify and/or adapt for various applications such specific embodiments, without undue experimentation, without departing from the general concept of the present invention. Such adaptations and modifications are therefore intended to be within the meaning and range of equivalents of the disclosed embodiments, based on the teaching and guidance presented herein.

What is claimed is:

1. An article of clothing accommodating a pre-existing attire of a wearer, the article of clothing having a first front side connected to a back side connected to a second front side, the article of clothing accommodating a pre-existing medical attachment connected to the wearer, the article of clothing comprising:
a bottom hem extending around a bottom portion of the first front side, a bottom portion of the second front side, and a bottom portion of the back side;
a collar extending around a top portion of the first front side, a top portion of the second front side, and a top portion of the back side;
a first front placket on the first front side and extending from the collar to the bottom hem;
a second front placket on the second front side and extending from the collar to the bottom hem;
a first sleeve connected to and extending outward from the first front side and the back side, the first sleeve including a wrist hem at a distal end from the connection to the first front side and the back side;
a first lateral seam extending from the wrist hem to the first front placket at a position below the collar, the first lateral seam including: a top placket having a plurality of fasteners embedded therein and a bottom placket having a plurality of mating fasteners embedded therein;
wherein the wearer dons and removes the article of clothing accommodating the pre-existing medical attachment by opening the first lateral seam and wrapping the first sleeve around an arm of the wearer and closing the first lateral seem by engaging the plurality of fasteners in the top packet with the plurality of mating fasteners in the bottom packet, forming a medical attachment channel based on the spacing of the fasteners and the spacing of the mating fasteners, the top placket and the bottom packet.

2. The article of clothing of claim 1 further comprising:
a lateral seam closure connected to the first placket at a position below the first lateral seam; and
a lateral seam connector disposed on the collar, the lateral seam connector matingly receiving the lateral seam closure to secure the first front side to the collar in conjunction with the closing of the first lateral seam.

3. The article of clothing of claim 1 further comprising:
the first front placket having a plurality of front fasteners disposed therein;
the second front placket having a plurality of front mating fasteners disposed therein;
wherein engaging the plurality of front fasteners with the plurality of front mating fasteners dons the sweater around a front of the wearer without disruption of pre-existing medical device attachments.

4. The article of clothing of claim 1, the first sleeve including a pocket disposed therein.

5. The article of clothing of claim 1, wherein the pocket includes a lining accommodating at least one of: a heating packet and a cooling packet.

6. The article of claim of claim 1, the first sleeve including wiring for connecting at least one electrical connection.

7. The article of clothing of claim 6, wherein the electrical connection relates to a biofeedback sensor device.

8. The article of clothing of claim 1 further comprising:
a wrist pull tab connected to the wrist hem and disposed on at least one of the top placket and the bottom placket, the wrist pull tab having a tab fastener disposed thereon; and
a wrist tab mating fastener disposed on at least one of the top placket and the bottom placket, the wrist tab mating fastener disposition being on opposite of the lateral seam from the wrist pull tab;
wherein upon donning the sweater, the wrist pull tab connector closes the wrist hem by engaging the wrist tab mating fastener.

9. The article of clothing of claim 8 further comprising an interior channel accessible extending through the first sleeve and into at least one of the first front side and a back side, facilitating air flow therethrough.

10. The article of clothing of claim 1, wherein the article is reversible.

11. The article of clothing of claim 1 further comprising:
at least one wireless speaker disposed in the collar.

12. An article of clothing accommodating a pre-existing attire of a wearer, the article of clothing having a first front side connected to a back side connected to a second front side, the article of clothing accommodating a pre-existing medical attachment connected to the wearer, the article of clothing comprising:
a bottom hem extending around a bottom portion of the first front side, a bottom portion of the second front side, and a bottom portion of the back side;
a collar extending around a top portion of the first front side, a top portion of the second front side, and a top portion of the back side;
a first front placket on the first front side and extending from the collar to the bottom hem;
a second front placket on the second front side and extending from the collar to the bottom hem;
a first sleeve connected to and extending outward from the first front side and the back side, the first sleeve including a first wrist hem;
a first lateral seam extending from the first wrist hem to the first front placket at a position below the collar, the first lateral seam including: a first top placket having a plurality of first fasteners embedded therein and a first bottom placket having a plurality of first mating fasteners embedded therein;
a second sleeve connected to and extending outward from the second front side and the back side, the second sleeve including a second wrist hem;
a second lateral seam extending from the second wrist hem to the second front placket at a position below the collar, the second lateral seam including: a second top placket having a plurality of second fasteners embedded therein and a second bottom placket having a plurality of second mating fasteners embedded therein;
wherein the wearer dons and removes the article of clothing accommodating the pre-existing medical attachment by opening the first lateral seam and wrapping the first sleeve around an arm of the wearer and closing the first lateral seem by engaging the plurality of fasteners in the top packet with the plurality of mating fasteners in the bottom packet, forming a medical attachment channel based on the spacing of the fasteners and the spacing of the mating fasteners, the top placket and the bottom packet.

13. The article of clothing of claim 12 further comprising:
a first lateral seam closure connected to the first placket at a position below the first lateral seam; and
a first lateral seam connector disposed on the collar, the first lateral seam connector matingly receiving the first lateral seam closure to secure the first front side to the collar in conjunction with the closing of the first lateral seam.

14. The article of clothing of claim 13 further comprising:
a second lateral seam closure connected to the second placket at a position below the second lateral seam; and
a second lateral seam connector disposed on the collar, the second lateral seam connector matingly receiving the second lateral seam closure to secure the second front side to the collar in conjunction with the closing of the second lateral seam.

15. The article of clothing of claim 12 further comprising:
a first wrist pull tab connected to the first wrist hem and disposed on at least one of the first top placket and the first bottom placket, the first wrist pull tab having a tab fastener disposed thereon; and
a first wrist tab mating fastener disposed on at least one of the first top placket and the first bottom placket, the first wrist tab mating fastener disposition being on opposite of the first lateral seam from the wrist pull tab;
wherein upon donning the sweater, the first wrist pull tab connector closes the first wrist hem by engaging the first wrist tab mating fastener.

16. The article of clothing of claim 15 further comprising:
a second wrist pull tab connected to the second wrist hem and disposed on at least one of the second top placket and the second bottom placket, the second wrist pull tab having a second tab fastener disposed thereon; and
a second wrist tab mating fastener disposed on at least one of the second top placket and the second bottom placket, the second wrist tab mating fastener disposition being on opposite of the second lateral seam from the second wrist pull tab;
wherein upon donning the sweater, the second wrist pull tab connector closes the second wrist hem by engaging the second wrist tab mating fastener.

17. The article of clothing of claim 12, the first sleeve including a pocket disposed therein.

18. The article of claim of claim 12, the first sleeve including wiring for connecting at least one electrical connection.

19. The article of clothing of claim 18, wherein the electrical connection relates to a biofeedback sensor device.

20. The article of clothing of claim 12 further comprising:
at least one wireless speaker disposed in the collar.
